(19) United States
(12) Patent Application Publication
(10) Pub. No.: US 2023/0335255 A1
(43) Pub. Date: Oct. 19, 2023

(54) SYSTEMS AND METHODS FOR IMPROVING BEHAVIOR WITHIN A SHARED LIVING SPACE

(71) Applicant: UIPCO, LLC, San Antonio, TX (US)

(72) Inventors: Galo M. Alava, Tampa, FL (US); Amanda Michelle Boyd, Denver, CO (US); Ramsey Devereaux, San Antonio, TX (US); Gregory Mark Lamontagne, Helotes, TX (US); Elizabeth J. Rubin, San Antonio, TX (US); Brian Tougas, Spring Branch, TX (US); Courtney St. Martin, Fomey, TX (US); Michael Kyne, Saint Petersburg, FL (US)

(21) Appl. No.: 17/724,274

(22) Filed: Apr. 19, 2022

(51) Int. Cl. G16H 20/70; G06Q 40/08; H04L 67/52

(52) U.S. Cl. CPC G16H 20/70; G06Q 40/08; H04L 67/52

(57) ABSTRACT

The present disclosure is related to a method for a monitor system containing a processor that may receive position data from one or more sensors associated with one or more electronic devices. The position data may represent the devices' various locations over time. The monitor system may determine whether the locations represented by the position data correspond to expected locations generated by a predictive model. After determining that the locations and expected locations do not correspond to one another, the monitor system may send an alert to a computing device.

### CROSS-REFERENCE TO RELATED APPLICATION

This application claims priority to and benefit of U.S. Provisional Patent Application Ser. No. 63/176,667, titled “SYSTEMS AND METHODS FOR IMPROVING BEHAVIOR WITHIN A SHARED LIVING SPACE,” filed on Apr. 19, 2021, the entirety of which is incorporated by reference into the present disclosure.

### BACKGROUND

The present disclosure relates generally to systems and methods for incentivizing certain types of behavior with electronic devices. More specifically, the present disclosure relates to systems and methods for controlling risk of property loss or compromise of privacy in shared space living environments.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a method includes receiving, via a processor, position data from one or more sensors associated with one or more electronic devices. The position data is representative of one or more locations of the one or more electronic devices with respect to time. The method may also include determining, via the processor, whether the one or more locations of the one or more electronic devices correspond to one or more expected locations of the one or more electronic devices at one or more expected times based on the position data and a predictive model that includes a plurality of expected locations at a plurality of expected times. Additionally, the model includes sending, via the processor, an alert to a computing device in response to determining that the one or more locations of the one or more electronic devices do not correspond to the one or more expected locations at the one or more expected times.

In another embodiment, a system for monitoring devices in a shared living space includes one or more sensors configured to collect position data of a plurality of electronic devices, as well as a computing device configured to receive first position data from the one or more sensors. The first position data is representative of a first set of locations of a first electronic device of the plurality of electronic devices over a first amount of time. The computing device is also configured to receive second position data from the one or more sensors. The second position data is representative of a second set of locations of a second electronic device of the plurality of electronic devices over the first amount of time. The computing device is also configured to determine that the first electronic device and the second electronic device are located in a shared living space based on the first position data and the second position data, as well as generate a predictive model that includes a plurality of expected locations for the first electronic device, the second electronic device, or both within the shared living space based on the first position data, the second position data, or both in response to determining that the first electronic device and the second electronic device are located in the shared living space. Additionally, the computing device is configured to receive third position data from the one or more sensors, wherein the third position data is representative of a third set of locations of the first electronic device over a second amount of time, as well as and send an alert to an additional computing device based on the third position data and the predictive model.

In yet another embodiment, a tangible, non-transitory, computer-readable medium, includes computer-readable instructions that, when executed by one or more processors, cause the one or more processors to receive first device use data representative of a first set of activities performed by a first electronic device over a first period of time at a set of locations, as well as receive second device use data representative of a second set of activities performed by a second electronic device over the first period of time at the set of locations. The one or more processors may also determine that the first electronic device is underused during the first period of time at the set of locations based on the first set of activities and a threshold amount of activities, receive an indication that the first electronic device and the second electronic device are being transported to the set of locations, and send an alert to a computing device in response to determining that the first electronic device is underused and that the first electronic device and the second electronic device are being transported to the set of locations. The alert comprises instructions to leave the first electronic device in a secure area.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### DRAWINGS

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for receiving data concerning electronic devices from various sources, in accordance with embodiments described herein;

FIG. 2 a block diagram of components that may be part of a monitor system used within the system of FIG. 1, in accordance with embodiments described herein;

FIG. 3 illustrates a flow chart of a method for generating a predictive model tracking the positions of various devices within an environment, in accordance with embodiments described herein;

FIG. 4 illustrates a flow chart of a method for utilizing a predictive model to alert users regarding detected abnormalities in device positions, in accordance with embodiments described herein;

FIG. 5 illustrates a flow chart of a method for utilizing a predictive model to alert users regarding recommendations to safeguard against device theft, in accordance with embodiments described herein; and

FIG. 6 illustrates a flow chart of a method for utilizing a predictive model to alert users regarding recommendations to safeguard against device theft in unsecure areas, in accordance with embodiments described herein.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. One or more specific embodiments of the present embodiments described herein will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Electronic devices (e.g., cell phones, watches, laptops, etc.) have become almost household items in recent years. Because of this, electronic devices are now used in a variety of environments including shared living spaces that present certain challenges with regard to keeping these devices secure. Shared living spaces are areas where inhabitants take personal property and thus allow others to share access to each other's personal property. However, the inhabitants are not always familiar with one another, and, as such, the risk of theft, damage, or other dangers to the personal property exists. This risk can have certain ramifications in personal property insurance rate calculation. With this in mind, the advent of sensors in common areas that may work in conjunction with sensors on the electronic devices has made tracking personal property and the various risks associated with shared space living more feasible than ever. That is, it may be beneficial to provide systems and methods to utilize device data acquired from the electronic device and other sensor data to mitigate personal property risk in shared living spaces.

In some embodiments, data acquired from various sensors (e.g., cameras, RFID sensors, magnetic sensors, etc.) disposed within the shared living space may be used to monitor the locations of one or more devices in relation to the shared living space and each other over time. This location data may be used to generate a predictive model for device locations at different points in time during the day. A computing system may then monitor the locations of the one or more devices and detect any abnormalities (e.g., a laptop left in a common area while the owner is away) with regard to the location of the devices during certain time periods, with respect to the locations of other individuals, with respect to the location of other devices, or the like. In response to detecting an abnormality, the computing system may send a user an indication that his or her actions are putting the property at a higher risk of being stolen, misused, and the like as compared to moving the property to another location. The indication may also provide a list of action items for mitigating said risk. Based on the actions performed by the user after receiving the indication, the system may dynamically adjust an insurance rate or policy that covers the property and notify the user regarding the same.

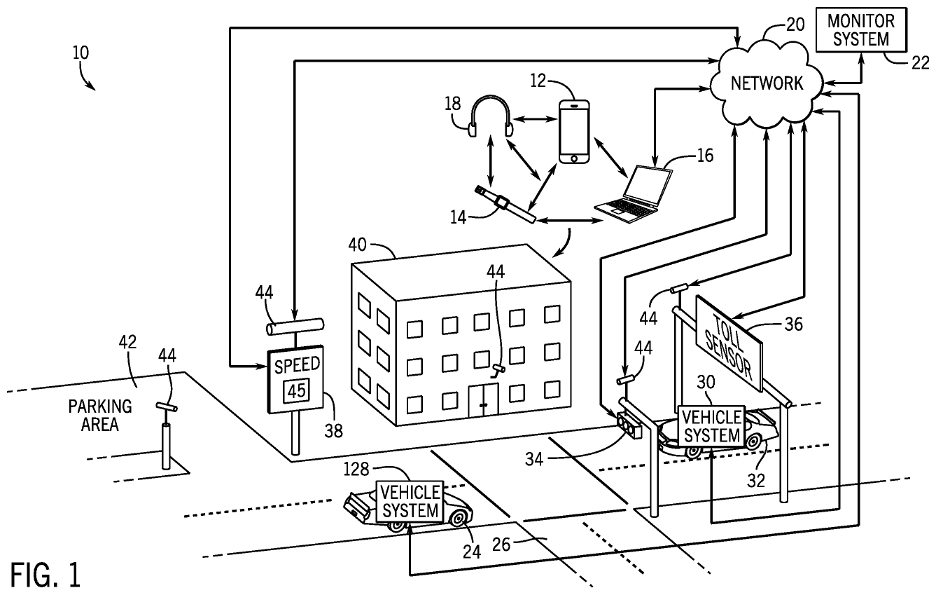

In other embodiments, sensors may be used to associate devices that are kept in close proximity of one another during certain periods of time. The computing system may monitor the movements and activities of the devices with respect to time to establish a pattern or expected grouping of devices during certain time periods. Specifically, the data may include a location and activity record of a group of devices (e.g., a laptop, cell phone, and smart watch) that regularly travel together and are used within certain time periods of each other (e.g., use of watch, phone, and laptop at employment site during expected employment hours). The computing system may monitor the locations and activities of these devices to generate a predictive model of the devices' locations and expected patterns of use. In some embodiments, the computing system may use the model to identify devices that may not regularly travel together. The computing system may send an alert to the user to leave these devices in a secure area, rather than regularly carrying them with other devices. Each of these embodiments will be described in detail below in FIGS. 1-6.

By way of introduction, FIG. 1 illustrates a block diagram of a system 10 in which certain electronic devices may communicate with each other. The system 10, for example, may include devices such as a mobile computing device 12, a wearable device 14, a laptop device 16, and an audio device 18. In certain embodiments, the devices may communicate with one another over a network 20. The network 20 may be any suitable wired or wireless (e.g., radio or light based) network that may facilitate communication of data between devices. In some embodiments, the network 20 may be a Wi-Fi network, a light detection and ranging (LIDAR) network, a LIDAR device, a 4G network, a 4G LTE network, a 5G network, a Bluetooth network, a Near Field Communication (NFC) network, or any suitable network for communicating information between devices. Communication via the network 20 may allow the devices, or any other available sensors connected to the network 20, to collect and communicate data pertaining to the location (e.g., position data) and use of the devices. This data may include location data, activity data, image data, audio data, temperature data, or a variety of other data types.

The collection of data may be provided to a monitor system 22, which may monitor data pertaining to the various devices over certain time periods. The monitor system 22 may be in communication with the network 20, in direct communication with the various devices, or in any other communication setup that allows it to monitor the devices and the data. In some embodiments, the monitor system 22 may be a remote computing system, a local computing system, one of the devices, or a number of other suitable computing devices. The monitor system 22 may also have the capability to process the data, and may, for example, generate predictive models representative of an expected behavior (e.g., applications usage, battery consumption rate, network usage, location) of the device, generate analytics data pertaining to the data, and the like. In one embodiment, the monitor system 22 may process data concerning device positions with respect to time to generate data concerning an expected velocity and acceleration of the devices during different time periods.

The monitor system 22 may also connect, via the network 20, to a variety of other sensors contained in the system 10 to collect additional data regarding an environment surrounding the devices. That is, the system 10 may include a variety of systems and sensors that can provide data concerning the devices. Several of these sensors and systems may be found in the infrastructure of the environment where the user carries the devices. For instance, the user may travel in a vehicle 24. The vehicle 24 may be a car, a public transport vehicle, such as a bus, rail car, or subway, a motorcycle, a bicycle, or any other type of vehicle used to navigate infrastructure 26 of the environment. Additionally, the vehicle 24 may include a variety of devices used in vehicle function and navigation, such as an accelerometer, a Global Positioning System (GPS) receiver, or another device.

The infrastructure 26 may be a road, a rail line, a subway line, a bike path, or any other type of infrastructure conducive to transportation via the vehicle 24. The vehicle 24 may also contain a vehicle system 28 that is capable of communicating with the various types of devices. The vehicle system 28 may include a processor or any device capable of facilitating communication with the various devices via a suitable wired or wireless protocol. As such, the vehicle system 28 may collect different types of data via the devices. In one embodiment, a device such as the mobile computing device 12 is carried into the vehicle 24 and communicatively connects (e.g., via Bluetooth) to the vehicle system 28. The vehicle system 28 may also communicate directly with the wearable device 14 or via the network 20. In some embodiments, the vehicle system 28 may collect data pertaining to the location and activity of the mobile computing device 12 and communicate the data to the monitor system 22 via the network 20. The vehicle system 28 may also communicate with other vehicle systems, such as a vehicle system 30 in vehicle 32. In this way, the vehicle system 28 to obtain traffic data from the vehicle system 30, as well as data pertaining to the use and location of other devices in communication with the vehicle system 30.

The vehicle system 28 may also communicate with other infrastructure systems and sensors on the infrastructure 26. For instance, a traffic light 34 may communicate with the vehicle system 28. The traffic light 34 may be any suitable traffic device that illuminates different lights (e.g., light-emitting diode lights) to convey traffic commands (e.g., go, stop, slow). In addition to illuminating different lights, the traffic light 34 may send data (e.g., time to next green/red/yellow light, duration of next green/red/yellow, switching to blinking red/yellow in a certain amount of time, switching to red, green or yellow in a certain amount of time, mean/median speed of cars thru intersection over a period of time) or a log that is indicative of the state of the traffic light 34 at various times to the monitor system 22 or any other suitable device via the network 20, via a direct communication link, or the like.

The vehicle system 28 may also communicate with a toll sensor 36. The toll sensor 36 may detect the presence of a tag (e.g., radio frequency identification tag) or an identifier used to access a toll road. The tag may be associated with a driver, a passenger, a physical item, a vehicle, a smart device, and the like. The toll sensor 36 may acquire data regarding the owner of a respective tag and may transmit data related to the detected tag to the monitor system 22 or any other suitable device via the network 20, via a direct communication link, or the like. The data may include a time stamp, speed, direction, weather conditions, traffic volume, and other properties associated with the detected tag, a vehicle owner associated with the tag, a vehicle make/model/year associated with the tag, and the like.

The vehicle system 28 may also communicate with a speed detector 38. The speed detector 38 may detect a speed of a vehicle using radar, image data, or any suitable speed detecting technology. The speed detector 38 may display the detected speed on an integral display and may transmit data related to the detected speed to the monitor system 22 or any other suitable device via the network 20, via a direct communication link, a broadcast signal, or the like. The data may include the detected speed, a time stamp associated with the detected speed, and the like.

The system 10 contains a shared living space 40. The shared living space 40 is a space in which a variety of inhabitants live alongside one another (e.g., army barracks, a college dorm, a hostel, a shelter, a hospital or other health facility, a boarding school, and the like). The inhabitants of the shared living space 40 may own a variety of devices, each of which may be used in different areas of the shared living space 40. Additionally, the system 10 may contain a parking area 42. The parking area 42 may be a parking lot, parking garage, car storage tower, bus stop, rail car stop, subway station, bicycle rack, or any other temporary or long term vehicle storage location. The parking area 42 may be a place for the inhabitants of shared living space 40 to enter and exit in a vehicle such as vehicle 24 or vehicle 32.

In certain embodiments, one or more of the traffic devices described above may be accompanied by a respective camera 44. The camera 44 may be any suitable image sensor, such as a still image sensor, a video image sensor, an infrared image sensor, a thermal image sensor, a light sensor, or the like. The camera 44 may send image data (e.g., emergency vehicle identified, oversized vehicle identified, school bus identified, stolen vehicle identified, unregistered vehicle identified, uninspected/expired-inspection vehicle identified, military vehicle identified, crowd on or near road identified, accident identified, bicycle(s) identified, etc.) collected at different times to the monitor system 22 or any other suitable device via the network 20, via a direct communication link, or the like. In some embodiments, the transmission of the image data may be coordinated with the transmission of the respective data from the respective traffic device. For example, the speed detector 38 may send speed data of the vehicle 24 and the camera 44 collocated with the speed detector 38 may send the image data that corresponds to the speed data to the monitor system 22, such that the monitor system 22 may use the image data to verify the speed data. To coordinate the transmission of data, each respective device may broadcast a signal indicating that it will transmit its collected data for a certain time period to the monitor system 22. After receiving the broadcast signal, other devices may also send its collected data for the same time period to the monitor system 22.

In some embodiments, the monitor system 22 may collect data pertaining to the mobile computing device 12, wearable device 14, laptop device 16, and audio device 18 as they are used in the system 10 throughout a time period. The monitor system 22 may collect supplemental data about device use and location in the shared living space 40, in the vehicles 24 and 32, and along the infrastructure 26 by communicating with the vehicle systems 24 and 28, the traffic light 34, the toll sensor 36, the speed detector 38, the camera 44, and other devices or sensors. The monitor system 22 may use the collected data to generate predictive data concerning the location and user of the devices. In one embodiment, the monitor system 22 may use data collected from the systems and devices listed above to create a training data set. The monitor system 22 may then use the training data set generate a predictive machine learning model. The predictive machine learning model may predict device location and activity over a set time period, and can be used for risk assessment purposes.

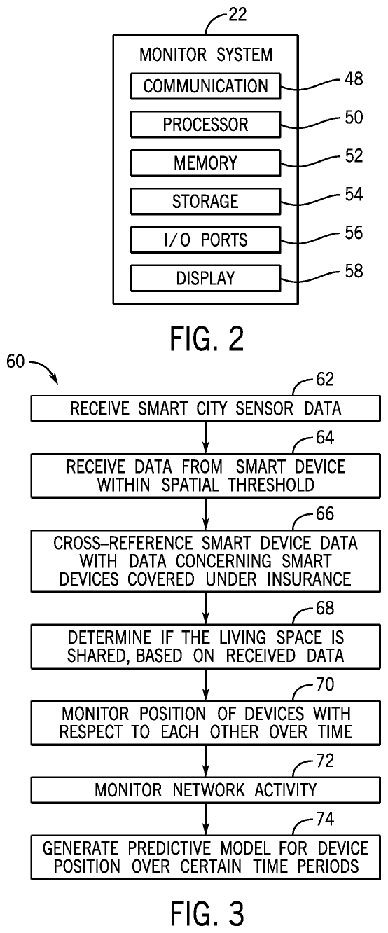

Now turning to the next figure, FIG. 2 illustrates a block diagram of example components within the monitor system 22. For example, the monitor system 22 may include a communication component 48, a processor 50, a memory 52, a storage 54, input/output (I/O) ports 56, a display 58, and the like. The communication component 48 may be a wireless or wired communication component that may facilitate communication between the monitor system 22, the traffic devices, the network 20, and the like. Additionally, the communication component 48 may facilitate data transfer to monitor system 22, such that monitor system 22 may receive data from the other components depicted in FIG. 1 and the like.

The processor 50 may be any type of computer processor or microprocessor capable of executing computer-executable code. The processor 50 may also include multiple processors that may perform the operations described below.

The memory 52 and the storage 54 may be any suitable articles of manufacture that can serve as media to store processor-executable code, data, or the like. These articles of manufacture may represent computer-readable media (e.g., any suitable form of memory or storage) that may store the processor-executable code used by the processor 50 to perform the presently disclosed techniques. The memory 52 and the storage 54 may also be used to store data described, various other software applications for analyzing the data, and the like. The memory 52 and the storage 54 may represent non-transitory computer-readable media (e.g., any suitable form of memory or storage) that may store the processor-executable code used by the processor 50 to perform various techniques described herein. It should be noted that non-transitory merely indicates that the media is tangible and not a signal.

The I/O ports 56 may be interfaces that may couple to other peripheral components such as input devices (e.g., keyboard, mouse), sensors, input/output (I/O) modules, and the like. The display 58 may operate to depict visualizations associated with software or executable code being processed by the processor 50. In one embodiment, the display 58 may be a touch display capable of receiving inputs from a user of the monitor system 22. The display 58 may be any suitable type of display, such as a liquid crystal display (LCD), plasma display, or an organic light emitting diode (OLED) display, for example. Additionally, in one embodiment, the display 58 may be provided in conjunction with a touch-sensitive mechanism (e.g., a touch screen) that may function as part of a control interface for the monitor system 22.

It should be noted that the components described above with regard to the monitor system 22 are exemplary components and the monitor system 22 may include additional or fewer components as shown. Additionally, it should be noted that the various computing devices described above with reference to FIG. 1 may also include similar components as described as part of the monitor system 22.

With the foregoing in mind, FIG. 3 illustrates a flow chart of a method 60 for generating a predictive model for device position over certain time periods. Although the following description of the method 60 is described in a particular order, it should be noted that the method 60 is not limited to the depicted order; and, instead, the method 60 may be performed in any suitable order. In addition, although the monitor system 22 is described as performing the method 60, it should be understood that the method 60 may be performed by any suitable computing device including, for instance, the mobile computing device 12 and the like.

Referring now to FIG. 3, at block 62, the monitor system 22 may receive data collected from the various sensors and systems described in FIG. 1. The data, as described above, may pertain to device location and activity, traffic data, vehicle data, and the like. This data may be referred to as smart city data and may represent aggregate data related to patterns and trends in a community. As such, the smart city data may correspond to traffic conditions in an area, a number of pedestrians in the area, image data related to individuals entering or exiting a building, and other aggregate data that characterize observable data within an area.

At block 64, the monitor system 22 may receive data from devices associated with a particular user that are located within a certain spatial threshold. This data may be a set of device identifications (IDs), a set of device internet protocol (IP) addresses, information concerning the types of devices corresponding to the data, device serial numbers or product numbers, and the like. Devices may communicate this data by way of a Wi-Fi network, a 4G network, a 4G LTE network, a 5G network, a Bluetooth network, a near field communication (NFC) network, a local area network (LAN), or any other means of data communication. The spatial threshold may be defined as an area surrounding a chosen device, a predetermined location, or any space useful for the analysis described below. In one embodiment, a user's devices, as well as the devices owned by other inhabitants of a share living space, may send data to monitor system 22.

At block 66, the monitor system 22 may cross-reference the received device data with insurance data. This insurance data may be data detailing information concerning the user, personal property insurance coverage for the user, an insurance rate for the user or for the user's property, the data types detailed in the paragraph above, and the like. In certain embodiments, the insurance data may concern a group insurance plan that includes data for a group of members. Cross referencing this data with device data received within a shared living space may allow the monitor system 22 to determine whether the user possesses devices covered under the insurance plan.

At block 68, the monitor system 22 may determine if the living space in which the devices are detected is shared. For example, the monitor system 22 may examine the insurance policy identified at block 66 to determine whether the living space is cohabitated by separate users that have devices that are covered by the same policy. That is, the monitor system 22 may examine the insurance plan described above and determine whether the devices covered by the insurance plan are located in the shared living space. For instance, the devices may have communication components that enable them to send data or identify themselves with the monitor system 22. The monitor system 22 may check whether the device data received from the devices match the devices covered by the group policy. If the monitor system 22 determines that there are multiple devices present in the space that are not covered by the insurance policy the monitor system 22 may determine that the user is sharing the living space with others that may not be part of the user's family or trusted network.

Alternatively, the monitor system 22 may prompt the user to indicate whether the space is shared or not. The relation of shared living space inhabitants can have drastic effects on the risk of device theft or other illicit activities. For instance, if a user inhabits a living space alone and keeps his devices in the living space, the device will have a low risk of theft, hacking, or other dangers while the user is at home, or away from home with the space locked. However, the risk increases greatly if the user inhabits a living space shared by other inhabitants. If these inhabitants are family members or other individuals that share a common insurance plan with the user, the risk likely does not increase. If the inhabitants are non-family members or other individuals that do not share a common insurance plan with the user, the risk may increase. This risk increase may warrant analyzing user behavior to mitigate risky behavior in the shared living space, as described in the embodiments below.

At block 70, the monitor system 22 may monitor the position of the devices with respect to one another in the shared living space over time. In some embodiments, the monitor system 22 may track the location of each device as it travels throughout the day. For example, the monitor system 22 may monitor the location of the mobile computing device 12 as the user carries it around different locations in the shared living space 40, or to other locations via the vehicle 24. Additionally, the monitor system 22 may monitor a precise location of the mobile computing device 12 and identify different settings (e.g., home, school, work, public transport, airport, etc.) in which the mobile computing device 12 is present. As such, the monitor system 22 may utilize the systems and sensors described in FIG. 1 to gather information about the risks present in each setting. The monitor system 22 may also monitor the proximity of certain devices with respect to other devices owned by different users. For instance, the monitor system 22 may note that the laptop device 16 is within a proximity to other devices, such as the audio device 18. The monitor system 22 may detect proximity between devices using sensors on each device. The sensors may communicate over a short range network (e.g., a Bluetooth network, NFC, etc.) and send the monitor system 22 data concerning the distance between devices with respect to time. The monitor system 22 may also note that the device is around other electronic devices owned by separate users. Furthermore, it may determine if a device is in proximity to its owner by detecting a proximity between difference devices associated with the same owner that are likely to within a close proximity of the person, such as the wearable device 14.

Furthermore, at block 72, the monitor system may monitor network activity over time. This network activity may be any activity communicated through the network 20. The activity may include active device use (e.g., calling, texting, web browsing, application use, music streaming, etc.) or passive functions (e.g., automated network connection, location finding, data reception, etc.) that include transmitting and/or receiving data via the network 20. In one embodiment the monitor system 22 may use the activity data to identify device locations that are outside a proximity of a user. For example, a user leaving a laptop open in a common area while the user is away may increase the risk of theft or unauthorized use. In another example, the monitor system 22 may detect the use of the mobile computing device 12 in a first location as well as the operation of the vehicle 24 in a second location different from the first location. The monitor system 22 may determine that since no other devices owned by the user are in the vehicle 24, the user may not be in possession of the mobile computing device 12 or the vehicle 24. The monitor system 22 may identifying this behavior as being related to increase likelihood of theft of the device.

In some embodiments, network activity data may include information related to one or more networks that are accessed by the devices over time. The network activity data may thus be used to identify a location that corresponds to the user, expected locations that corresponds to the devices over time, and the like.

At block 74, the monitor system 22 may generate a predictive model for device position over a period of time. The model may be generated based on the data collected at block 70 and block 72, and may be used to predict device position and activity throughout a chosen time period. In a certain embodiment, the monitor system 22 may generate a predictive model based on collected position and activity data collected by the various systems and cameras connected to network 20, and the predictive model may predict the expected location of the devices during certain regular activities (e.g., going to work, attending class, attending a weekly barracks meeting, etc.). In some embodiments, the predictive model may be generated in response to determining that the devices are located within a shared living space. This predictive model may be used to detect aberrations in regular device use indicative of insurance risks.

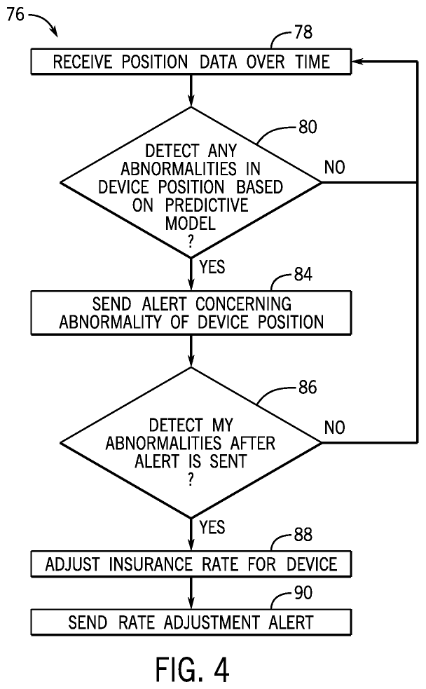

With this in mind, FIG. 4 depicts a method 76 for identifying abnormalities in expected device positions and expected device activities and adjusts user insurance rates based on the detected behavior. Although the following description of the method 76 is described in a particular order, it should be noted that the method 76 is not limited to the depicted order; and, instead, the method 76 may be performed in any suitable order. In addition, although the monitor system 22 is described as performing the method 76, it should be understood that the method 76 may be performed by any suitable computing device.

With this in mind, at block 78, the monitor system 22 may receive data concerning the device positions over time. In one embodiment, similar to the actions described at block 70 and block 72, the systems and cameras depicted in FIG. 1 may communicate via the network 20 to the monitor system 22 to monitor the device positions over time. It should be appreciated, however, that the method 76 may operate using tools and systems not found in FIG. 1.

At block 80, the monitor system 22 may detect abnormalities in the device positions over time based on the predictive model described at block 74. An abnormality may be any aberrance from the predicted path described at block 74. For example, the monitor system 22 may define an abnormality as a device moving outside a certain range that the predictive model expected the device would be during a certain time of day (e.g., the mobile computing device 12 is found outside of the user's workplace during working hours). In one embodiment, if a user takes a device away from its usual course for any reason (e.g., a vacation, an emergency situation, etc.), the user may alert the monitor system 22, via a smart phone application or the like, that an aberration will take place and the monitor system 22 will not record the aberration as an abnormality.

At block 80, if the monitor system 22 does not detect an abnormality, the monitor system 22 returns to block 78 and the process continues until the monitor system 22 detects an abnormality. However, if the monitor system 22 does detect an abnormality, the process progresses to block 84.

At block 84 the monitor system 22 may send an abnormality alert to the owner of the device that has departed from the predicted path. The alert may be communicated through the device itself, a telephone call, a notification depicted on a website associated with an account (e.g., insurance for the user, an email, or any other suitable means. The monitor system 22 may communicate the alert in response to a single abnormality, or in response to a number of abnormalities above a set abnormality threshold. By way of example, a user may bring the wearable device 14 to his workplace near his shared living space every day. Based on a predictive model, the monitor system 22 may expect the wearable device 14 to be in the user's office during working hours. Since the user may go out for lunch, the predictive model allows for one abnormality per day at the time the user usually leaves the building for lunch to a different location. If the user leaves the office for lunch, the monitor system 22 may not send an alert to the user, since it expects an abnormality at that time. If the user accidentally leaves the wearable device 14 in their car in the morning, however, the monitor system 22 may detect the abnormality and send the user an alert because the expected position of the wearable device 14 does not match its current location. The alert may notify the user that the device is not located in an expected location, warn the user of risks associated with leaving the device at its current location, prompt the user to indicate that a new routine has been added to their day, or any other notification of use.

After sending the alert, at block 86, the monitor system 22 may determine whether the detected abnormality remains or whether additional abnormalities are detected to examine the user's response to the alert. For example, the monitor system 22 may receive additional position data (e.g., corresponding to additional locations) indicative of a device's position at an expected time after the alert is sent. If the abnormalities are no longer present (e.g., the user retrieved the device), the monitor system 22 may return to block 78 to continue monitoring the device's activity and location. In one embodiment, the monitor system 22 may return to block 78 after the user corrects for any detected abnormalities within a corresponding time period. If the monitor system 22 continues to detect the same or additional abnormalities, the monitor system 22 may continue to block 88.

At block 88, the monitor system 22 may adjust the user's insurance rate based on the location abnormalities of the device. As referenced at block 66 in FIG. 3, the owner of a device may have a personal property insurance policy for the device. The policy may have a specific rate proportional to an assumed risk of damage or theft to the device with respect to the device's location. For instance, a user may have an insurance policy for the laptop device 16. The monitor system 22 may detect abnormalities in device location as described at block 80 and block 82, and the monitor system 22 may the send an abnormality alert as shown at block 84. In response to detecting another abnormality after sending the alert, the monitor system 22 may then adjust the insurance rate on the policy associated with the laptop device 16 based on an increased risk of theft and damage associated with the unresolved abnormalities. The rate may increase in response to one post-alert abnormality or a set number of acceptable post-alert abnormalities. The margin of increase for the insurance rate may be based on the circumstances of the post-alert abnormality or abnormalities (e.g., the number of abnormalities, the duration of the abnormality, the distance the device strays). The margin of increase may also be based on factors pertaining to the location of the device (e.g., crime rate data for the area, suspicious activity by nearby devices, time of day, distance between device and user, etc.). These factors may indicate greater risk of theft and damage, as before, but may also indicate risk of less tangible dangers (e.g., hacking). The insurance rate may also decrease in response to risk mitigating behavior (e.g., promptly preventing abnormalities in response to an alert, keeping devices close to the user, keeping devices in secure areas, etc.). In some embodiments, the monitor system may adjust the insurance rate of the policy based on a determination that the devices are located in a shared living space.

At block 90, the monitor system 22 may send a rate adjustment alert to the user. The system may send the alert in response to adjusting the rate. Similarly to block 84, the alert may be sent through a number of different mediums and may cause the recipient device to automatically execute an application that presents a visualization that represents the adjustment to the rate. In this way, the monitor system 22 may provide a real-time notification regarding insurance rate changes due to detected behavior. In addition, by sending the notification after detecting the abnormalities discussed above, the present embodiments may limit that network bandwidth used to transmit notifications to a computing device. That is, the notification may be transmitted after detecting the abnormalities and a threshold amount of change has been made to the insurance policy. As a result, the user may not be inundated with notifications regarding the detected abnormalities and possible insurance rate changes. Instead, the user may be notified in limited instances to preserve the processing capabilities of the user's computing device and the network bandwidth used to receive and transmit data.

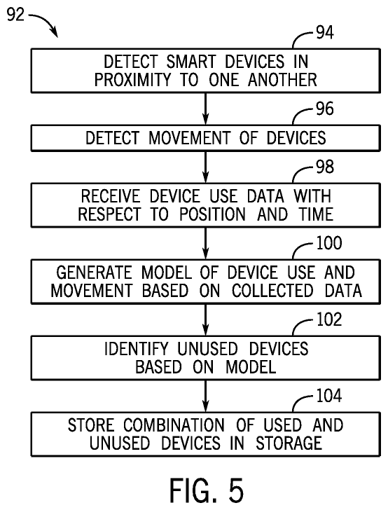

FIG. 4 describes a method in which the monitor system 22 may monitor user behavior to discourage abnormalities in electronic device use that may increase risk of theft or damage. This method may benefit both the user and the insurance provider by eliminating insurance claims caused by abnormal behavior. However, there are some circumstances in which normal, recurring user behavior may increase risk of device theft or damage. FIG. 5 describes a method for detecting such risky recurring behaviors.

FIG. 5 details method 92, which is a process for identifying used and unused devices in a group of devices in close proximity to one another using a predictive model. Although the following description of the method 92 is described in a particular order, it should be noted that the method 92 is not limited to the depicted order; and, instead, the method 92 may be performed in any suitable order. In addition, although the monitor system 22 is described as performing the method 92, it should be understood that the method 92 may be performed by any suitable computing device.

With this in mind, at block 94, the monitor system 22 may detect multiple devices in close proximity to one another. The monitor system 22 may define a pair of devices as being in close proximity with each other if they are within a set distance of each other, if they are near enough to directly communicate with one another (e.g., via NFC, Bluetooth, etc.), or the like.

At block 96, the monitor system 22 may detect device movement as described above. The system may detect movement similarly to the process at block 70 of FIG. 3. That is, the monitor system 22 may monitor the position of each device with respect to time, monitor device velocity, monitor device acceleration, and the like based on sensor data, analysis of the device's position data, and the like. The monitor system 22 may also monitor other factors, such as device activity.

At block 98, the monitor system 22 may receive data concerning device activity with respect to position and time. This activity may include active functions of the device, as well as passive functions. In an active function, the user may send an input to the device, which may prompt the device to generate visualizations in response to the user input. Active functions may also involve an amount of CPU processing above a certain threshold. Examples of active functions may include web browsing, audio streaming, telecommunication, application use, and the like. Passive functions may involve no user input whatsoever, and may involve an amount of CPU processing below the threshold. Examples of passive functions may include sleep modes, receiving text messages, displaying lock screens, and the like. For example, the monitor system 22 may monitor position and activity for the laptop device 16, the mobile computing device 12, and the wearable device 14 as the user takes them to class every school day. The monitor system 22 may recognize the devices as being in close proximity to each other based on whether each device is within a radius of six feet. The monitor system 22 may then monitor the position of the group of devices, as well as their activities over time. In this way, the monitor system 22 may identify which devices are in use at different times of day and at different locations. For instance, the devices may all stay within six feet of the user all day. However, the user may not actively or passively use laptop device 16, may actively and passively use the mobile computing device 12, and may passively use but not actively use the wearable device 14.

At block 100, the monitor system 22 may use the data collected at blocks 96 and 98 to generate a predictive model of device use and movement. Similar to the predictive model described at block 74 of FIG. 3, the predictive model at block 100 may be used to forecast both the locations and activities of a set of devices within a proximity to each other with reference to time. For example, a model may predict that a user will bring the mobile computing device 12 and the laptop device 16 to a class near a shared living space every Monday and heavily use the mobile computing device 12, but not use the laptop device 16. A scenario like this may indicate that the user is exposing her property to unnecessary risk, since the user is bringing the laptop device 16 into a non-secure area like a classroom where she is not expected to use the laptop device 16 according to the model. In the context of personal property risk mitigation, it may be beneficial to identify devices that do not see use on such expected trips.

At block 102, the monitor system 22 identifies unused or underutilized devices based on the predictive model. The monitor system 22 may determine that a device is unused or underutilized in several ways. The monitor system 22 may note that the device is powered off for the duration of a trip. The monitor system 22 may also detect passive function in the device, but no active function indicative of the user making use of the device. The monitor system 22 may also categorize various scopes of use for a device. For example, the monitor system 22 may define a device as underutilized based on the device remaining in one location when other devices travel, the device is actively in use (e.g., powered on, threshold amount of CPU processing in use) for less than five minutes when moved from a certain location (e.g., home), and the like. In another embodiment, the monitor system 22 may classify a device as being useful or active during trips based on whether the the device is actively used for a portion (e.g., threshold percentage) of the time during a particular trip.

At block 104, the monitor system 22 may store data detailing whether each device is actively in use or underutilized during a regular trip. This data may take the form of a binary value indicating whether the device is used or unused, a scaled value indicating a degree of frequency (e.g., portion of an amount of time) that the device is used, and the like. This scaled value may denote the percentage of total trips on which the device is used, the time percentage of each trip in which the device is used, or the like. The data may be stored in the storage 54 shown in FIG. 2, a cloud storage platform, or any other means of storage internal or external to the system being used.

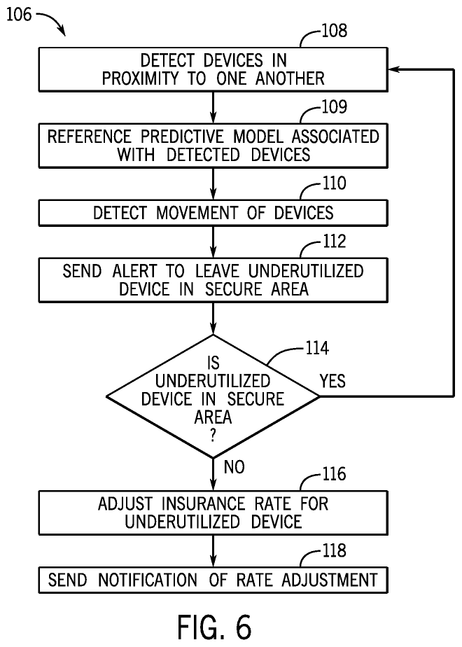

FIG. 5 describes the method for generating a predictive model of the location of devices in close proximity to one another over time, as well as whether each device is being used or underutilized. FIG. 6 describes a method for utilizing this predictive model to encourage users to leave underutilized devices in safe locations.

Now turning to FIG. 6, the illustrated method 106 is a method for sending an alert to a user indicating that a device is underutilized on a regularly scheduled trip, and adjusting an insurance rate based on the user's responsive behavior. Although the following description of the method 106 is described in a particular order, it should be noted that the method 106 is not limited to the depicted order; and, instead, the method 106 may be performed in any suitable order. In addition, although the monitor system 22 is described as performing the method 106, it should be understood that the method 106 may be performed by any suitable computing device.

With this in mind, at block 108 the monitor system 22 may detect devices that are located within a proximity to one another. This process may be similar to the one described at block 94 of FIG. 5.

At block 109, the monitor system 22 may reference a predictive model associated with the devices. The previously generated predictive model may be the predictive model described at block 100. The monitor system 22 may determine that the devices are associated with the predictive model based on the combination of the devices detected in close proximity to one another at block 108.

At block 110, the monitor system 22 may detects movement of the identified devices. This movement, as well as a time and date of the movement, may indicate to the monitor system 22 that a trip associated with a previously generated predictive model is beginning. The monitor system 22 may identify the predictive model based on previously collected data. Using the predictive model, the monitor system 22 may identify devices that are expected to be used and underutilized during the trip. The monitor system 22 may propose or generate a suggestion for the user to consider to minimize risk for property theft or damage during the expected trip.

At block 112, based on the detected movement of the devices to a predicted location, the monitor system 22 may send an alert to a computing device associated with the user to leave a particular device in a secure area. That is, the monitor system 22 may notify the user via the computing device that the particular device is rarely used (e.g., underused) in the predicted location. A device may be considered to be rarely used (e.g., underused) if the device is utilized under a threshold time value over a period of time. This alert, similarly to the alert described at block 84 in FIG. 4, may be sent to the user in a variety of means (e.g., telephone call, email, account notification, etc.) by way of a variety of devices, including one of the devices the user possesses. The user may set a preference for the one or more means by which the system communicates the alert. By way of example, the monitor system 22 may recognize that the user does not use the audio device 18 that the user brings to work every weekday. The monitor system 22 may then send an alert to the computing device associated with the user notifying him of the risks posed by bringing the particular device on a trip when the device is unlikely to be used. The alert may also suggest one or more possible action plans to mitigate the risk. The possible action plans may include leaving the device in a secure place (e.g., a dorm room, a safe, a space owned by a trusted acquaintance, etc.). The monitor system 22 may also provide the option to purchase an insurance policy for the device if the device is not covered by an existing policy. In another embodiment, the monitor system 22 may detect that another device is expected to be present on a trip according to the predictive model. In this scenario, the monitor system 22 may send an alert to the computing device of the user notifying them of the missing device.

At block 114, the monitor system 22 may check if the user heeded the alert at block 112. That is, if the user follows the instructions given by the alert, for instance, leaving an unused device in a secure area, the monitor system 22 returns to block 108 and continues. If the monitor system 22 detects that the user did not heed the alert (e.g., does not leave the unused device in a secure area), the monitor system 22 continues to block 116.

At block 116, the monitor system 22 may adjusts the insurance rate for the device based on a user's behavior. For example, the monitor system 22 may raise the insurance rate for the device if the user did not leave the device in the secure area. Similar to block 88 of FIG. 4, this rate adjustment applies to a personal property insurance policy associated with the device. The rate may increase if the user did not heed the alert described at block 112.

At block 118 the monitor system 22 may send a notification to a computing device associated to the user indicating that the monitor system 22 has adjusted the insurance rate for the device. This process is similar to the one described at block 90 of FIG. 4. For example, if a user receives an alert, such as the one described at block 112 to leave an unused device in a secure place, but the user does not comply and continues to bring the device on future trips, the monitor system 22 may increase the personal property insurance rate associated with the device.

In the embodiments described above, a monitor system utilizes smart city data to monitor electronic devices in a shared living space. The monitor system may receive data concerning device location, use, proximity to other devices, and other factors. The monitor system may utilize this data to generate predictive models describing patterns of device use as a user goes about regularly scheduled or recurring activities. The monitor system may use a predictive model to identify if a user is partaking in behavior that may increase the likelihood that a device is stolen, lost, damaged, or the like. The monitor system may then send an alert to a device associated with the user to change said behavior. If the user does not change their behavior, the monitor system may increase the insurance rates. The systems and methods described above benefit both the user and the insurance provider by encouraging user behaviors that result in fewer losses, and therefore fewer insurance claims.

While only certain features of disclosed embodiments have been illustrated and described herein, many modifications and changes will occur to those skilled in the art. It is, therefore, to be understood that the appended claims are intended to cover all such modifications and changes as fall within the true spirit of the present disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method, comprising:
receiving, via a processor, position data from one or more sensors associated with one or more electronic devices, wherein the position data is representative of one or more locations of the one or more electronic devices with respect to time;
determining, via the processor, whether the one or more locations of the one or more electronic devices correspond to one or more expected locations of the one or more electronic devices at one or more expected times based on the position data and a predictive model comprising a plurality of expected locations at a plurality of expected times; and
sending, via the processor, an alert to a computing device in response to determining that the one or more locations of the one or more electronic devices do not correspond to the one or more expected locations at the one or more expected times.

2. The method of claim 1, wherein the alert is configured to cause the computing device to display a notification indicating that the one or more locations do not correspond to the one or more expected locations.

3. The method of claim 1, wherein the predictive model is generated based on additional position data acquired from the one or more sensors associated with the one or more electronic devices over a period of time.

4. The method of claim 3, wherein the predictive model is generated based on network activity data associated with the one or more electronic devices, wherein the network activity data comprises a record of one or more networks accessed by the one or more electronic devices over time.

5. The method of claim 3, comprising:
receiving, via the processor, network activity data associated with the one or more electronic devices, wherein the network activity data corresponds to one or more expected network activities of the one or more electronic devices at the one or more expected times based on the predictive model, wherein the predictive model comprises the one or more expected network activities at the one or more expected times; and
sending, via the processor, an additional alert to the computing device in response to determining that the network activity data of the one or more electronic devices does not correspond to the one or more expected network activities.

6. The method of claim 1, comprising:
receiving, via the processor, additional position data from the one or more sensors after sending the alert, wherein the additional position data is representative of one or more additional locations of the one or more electronic devices with respect to time;
determining, via the processor, whether the one or more additional locations correspond to one or more additional expected locations of the one or more electronic devices at one or more additional expected times based on the additional position data and the predictive model; and
adjusting, via the processor, an insurance rate associated with the one or more electronic devices in response to the one or more additional locations failing to correspond to the one or more additional expected locations.

7. The method of claim 6, wherein the one or more additional locations match the one or more locations.

8. The method of claim 6, comprising sending, via the processor, an additional alert to the computing device indicative of the adjustment to the insurance rate associate with the one or more electronic devices, wherein the additional alert is configured to automatically present a visualization comprising the additional alert.

9. A system for monitoring devices in a shared living space, comprising:
one or more sensors configured to collect position data of a plurality of electronic devices;
a computing device configured to:
receive first position data from the one or more sensors, wherein the first position data is representative of a first set of locations of a first electronic device of the plurality of electronic devices over a first amount of time;
receive second position data from the one or more sensors, wherein the second position data is representative of a second set of locations of a second electronic device of the plurality of electronic devices over the first amount of time;
determine that the first electronic device and the second electronic device are located in a shared living space based on the first position data and the second position data;
generate a predictive model comprising a plurality of expected locations for the first electronic device, the second electronic device, or both within the shared living space based on the first position data, the second position data, or both in response to determining that the first electronic device and the second electronic device are located in the shared living space;
receive third position data from the one or more sensors, wherein the third position data is representative of a third set of locations of the first electronic device over a second amount of time; and
send an alert to an additional computing device based on the third position data and the predictive model.

10. The system of claim 9, wherein the computing device is configured to:
determine that the third set of locations fails to correspond to the plurality of expected locations provided by the predictive model; and
send the alert to the computing device in response to determining that the third set of locations fails to correspond to the plurality of expected locations.

11. The system of claim 9, wherein the predictive model is generated based on network activity data associated with the first electronic device, the second electronic device, or both, wherein the network activity data is indicative of one or more networks accessed by the first electronic device, the second electronic device, or both over the first amount of time.

12. The system of claim 9, wherein the additional computing device is associated with a user that corresponds to the first electronic device, the second electronic device, or both.

13. The system of claim 9, wherein the computing device is configured to display a message on a display prompting a user to confirm that the first electronic device and the second electronic device are located in the shared living space in response to determining that the first electronic device and the second electronic device are located in the shared living space.

14. The system of claim 9, wherein the alert is configured to cause the additional computing device to display a notification indicating that the third set of locations do not correspond to the plurality of expected locations.

15. The system of claim 9, wherein the computing device is configured to adjust an insurance policy in response to determining that the first electronic device and the second electronic device are located in the shared living space.

16. A tangible, non-transitory, computer-readable medium, comprising computer-readable instructions that, when executed by one or more processors, cause the one or more processors to:
receive first device use data representative of a first set of activities performed by a first electronic device over a first period of time at a set of locations;
receive second device use data representative of a second set of activities performed by a second electronic device over the first period of time at the set of locations;
determine that the first electronic device is underused during the first period of time at the set of locations based on the first set of activities and a threshold amount of activities;
receive an indication that the first electronic device and the second electronic device are being transported to the set of locations; and
send an alert to a computing device in response to determining that the first electronic device is underused and that the first electronic device and the second electronic device are being transported to the set of locations, wherein the alert comprises instructions to leave the first electronic device in a secure area.

17. The computer-readable medium of claim 16, comprising computer-readable instructions that, when executed by the one or more processors, cause the one or more processors to:
determine that the first electronic device is located in the secure area after sending the alert; and
adjust an insurance rate associated with the first electronic device in response to determining that the first electronic device is located in the secure area.

18. The computer-readable medium of claim 16, comprising computer-readable instructions that, when executed by the one or more processors, cause the one or more processors to:
determine that the first electronic device is not located in the secure area after sending the alert; and
adjust an insurance rate associated with the first electronic device in response to determining that the first electronic device is not located in the secure area.

19. The computer-readable medium of claim 18, comprising computer-readable instructions that, when executed by the one or more processors, cause the one or more processors to send an additional alert to the computing device, wherein the additional alert is configured to cause the computing device to display a notification indicating that the insurance rate has been adjusted.

20. The computer-readable medium of claim 16, comprising computer-readable instructions that, when executed by the one or more processors, cause the one or more processors to store data indicative of the first electronic device being underused.
